(19) United States
(12) Patent Application Publication
(10) Pub. No.: US 2008/0045964 A1
(43) Pub. Date: Feb. 21, 2008

(54) DEVICE FOR CARTILAGE REPAIR

(76) Inventor: Allan Mishra, Menlo Park, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/838,737

(22) Filed: Aug. 14, 2007

Related U.S. Application Data
(60) Provisional application No. 60/838,064, filed on Aug. 16, 2006.

(51) Int. Cl. A61B 17/56; A61B 17/32

(52) U.S. Cl. 606/79; 606/167

(57) ABSTRACT

A surgical awl is disclosed for tissue repair. The surgical awl includes an internal lumen through which bioactive substances, such as platelet-rich plasma, may be delivered to a site of tissue or organ disease or dysfunction. In particular, the surgical awl is useful as a microfracture awl for cartilage repair augmented by delivery of platelet-rich plasma through the lumen of the device.

### CROSS-REFERENCE TO RELATED APPLICATIONS

This application claims the benefit under 35 U.S.C. § 119(e) of U.S. Provisional Patent Application No. 60/838,064, filed Aug. 16, 2006, entitled “Device for Cartilage Repair,” which is hereby incorporated by reference herein in its entirety.

### BACKGROUND

1. Field of the Invention

The invention relates to a device for cartilage repair and methods of using the device to repair cartilage and to deliver bioactive substances such as platelet-rich plasma to tissues.

2. Description of the Related Art

Current clinical treatments for symptomatic cartilage defects involve techniques aimed at: 1) removing surface irregularities by shaving and debridement 2) penetration of subchondral bone by drilling, fracturing or abrasion to augment the natural repair response 3) joint realignment or osteotomy to use remaining cartilage for articulation 4) pharmacological modulation 5) tissue transplantation and 6) cell transplantation (Newman, 1998; Buckwalter and Mankin, 1997). Most of these methods have been shown to have some short term benefit in reducing symptoms (months to a few years) while none have been able to consistently demonstrate successful repair of articular lesions after the first few years. The bone marrow-stimulation techniques of shaving, debridement, drilling, fracturing and abrasion athroplasty permit temporary relief from symptoms but produce a sub-functional fibrocartilagenous tissue that is eventually degraded. Pharmacological modulation supplying growth factors to defect sites can augment natural repair but to date insufficiently so (Hunziker and Rosenberg, 1996; Sellers et al., 1997). Allograft and autograft osteochondral tissue transplants containing viable chondrocytes can effect a more successful repair but suffer from severe donor limitations (Mahomed et al., 1992; Outerbridge et al., 1995).

One specific example of a cartilage disorder is a full thickness defect in the articular surface of a knee. This could occur with an acute injury or via chronic degeneration. Many different non-operative and operative treatments exist for this problem. Typically, rest, icing, physical therapy and some form of oral or injected medication is recommended. Braces and alternative forms of treatment such as acupuncture may also be tried. When these fail, surgery is offered as an option. The spectrum of surgical treatment options include: arthroscopic debridement and lavage, osteotomy, or total knee replacement. Another option is micro-fracture surgery.

Micro-fracture surgery involves using a solid awl to penetrate the subchondral bone that overlies a cartilage defect. The awl creates a hole or cavity in the bone that leads to the bone marrow underneath the cartilage defect. Repair of the cartilage defect using this technique is accomplished via the cells that leak out of the bone marrow. These bone marrow cells include bone hematopoetic and mesenchymal stem cells. These cells have the ability to transform into a variety of cell types including cartilage.

Unfortunately, micro-fracture surgery is only inconsistently efficacious. Successful results rely on a complete fill of the defect with a significant number of cells. If the fill of the defect is inadequate, the clinical results are typically either equivocal or even poor.

What is needed is a way to improve the ability of the body to fill that defect. If there is better defect fill, better clinical outcomes are expected. A technical problem addressed by the present invention is improvement in filling the cartilage defect.

### SUMMARY

An embodiment of a device comprises a tapered elongated member having a proximal end, a central longitudinal axis, and an awl shaped distal end. The proximal end comprises a handle for manipulation of the device and an access port comprising a luer lock on the side of the handle for the introduction of a fluid. The awl shaped distal end comprises an outlet port for discharge of the fluid. The access port is adapted to be in fluid communication with the outlet port through an internal channel disposed within the elongated member. The internal channel forms a hollow length extending throughout at least a part of the tapered elongated member, and the tip of the awl shaped distal end is angled with respect to the central longitudinal axis.

Another embodiment of a device comprises a tapered elongated member having a handle at a proximal end and a shaft comprising an awl shaped distal end. The elongated member comprises an internal channel disposed along a longitudinal axis within the elongated member. The internal channel forms a hollow length extending along at least a portion of the longitudinal axis, the internal channel having at least one input and an output at the awl shaped distal end.

An embodiment of a method of treating an injury, wear, or defect in an individual is disclosed. The method comprises (a) identifying an area of injury, wear or defect, and (b) inserting an embodiment of a device into the identified area. The device may comprise a tapered elongated member having a proximal end, a central longitudinal axis, and an awl shaped distal end. The proximal end comprises a handle for manipulation of the device and an access port comprising a luer lock on the side of the handle for the introduction of a fluid. The awl shaped distal end comprises an outlet port for discharge of the fluid. The access port is adapted to be in fluid communication with the outlet port through an internal channel disposed within the elongated member. The internal channel forms a hollow length extending throughout at least a part of the tapered elongated member, and the tip of the awl shaped distal end may be angled with respect to the central longitudinal axis. The method further comprises (c) creating a cavity in the area using the awl shaped distal end of the device; (d) attaching a delivery system to the access port for delivery of a bioactive substance; (e) delivering the bioactive substance through the internal channel and into the identified area; (f) closing the access port on the device; (g) compressing the bioactive substance into the identified area; and (h) removing the device.

An embodiment of a method of treating an injury, wear, or defect in an individual is disclosed. The method comprises (a) identifying an area of injury, wear or defect, and (b) inserting an embodiment of a device into the identified area. The device comprises a tapered elongated member having a handle at a proximal end and a shaft comprising an awl shaped distal end. The elongated member comprises an internal channel disposed along a longitudinal axis within the elongated member. The internal channel forms a hollow length extending along at least a portion of the longitudinal axis, the internal channel having at least one input and an output at the awl shaped distal end. The method further comprises (c) creating a cavity in the area using the awl shaped distal end of the device; (d) attaching a delivery system to the input for delivery of a bioactive substance; (e) delivering the bioactive substance through the internal channel and into the identified area; (f) closing the input of the device and/or stopping the delivery of the bioactive substance through the input channel; (g) compressing the bioactive substance into the identified area; and (h) removing the device.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows a perspective view of one embodiment of the microfracture awl.

FIG. 2 shows a perspective view of one embodiment of the awl tip.

FIG. 3a shows a view of one embodiment where the awl tip is pointed.

FIGS. 3b and 3c show views of one embodiment where the awl tip is flat-angled like a scraper.

FIG. 4a is a side view of one embodiment of the microfracture awl.

FIG. 4b is a cross-sectional view of one embodiment of the microfracture awl.

FIG. 5a shows a side view of one embodiment of the microfracture awl.

FIG. 5b shows a cross-sectional view of one embodiment of the microfracture awl.

FIG. 6 shows the effect of platelet-rich plasma (PRP) on mesenchymal stem cell proliferation. Mesenchymal stem cells were cultured in 10% unactivated buffered PRP for 7 days. Eight trials were performed. PRP showed higher density in all trials. Control Average=0.199; PRP Average=1.041. A five-fold increase was observed with the PRP treatments which is statistically significant (p<0.001).

FIG. 7 shows the increase in aggrecan mRNA, a cartilage marker, in the presence of PRP (p<0.001).

FIG. 8 shows the increase in SOX-9 mRNA, a cartilage marker, in the presence of PRP (p<0.001).

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

In one embodiment, the present invention is a device to deliver bioactive materials to an area of damaged cartilage in order to produce a better repair. The treated tissue may be connective tissue, cardiac muscle or tissue, spinal tissue (including nerves, spinal cord, disc or vertebral bodies), internal organs (including pancreas, lungs, liver, intestines, bladder or other solid organ tissue), skin tissue, brain tissue, vascular tissue (including veins, arteries or lymphatic tissue), ocular, ear, nose or throat tissue, or other hematologic, endocrine or integumentary tissue. In a preferred embodiment, the tissue is connective tissue. Connective tissue includes but is not limited to cartilage (articular and meniscus), ligament, tendons, fascia, bone and spinal tissue.

In another preferred embodiment, the awl may be used to deliver growth factors such as platelet-rich plasma, to the scalp to stimulate hair growth.

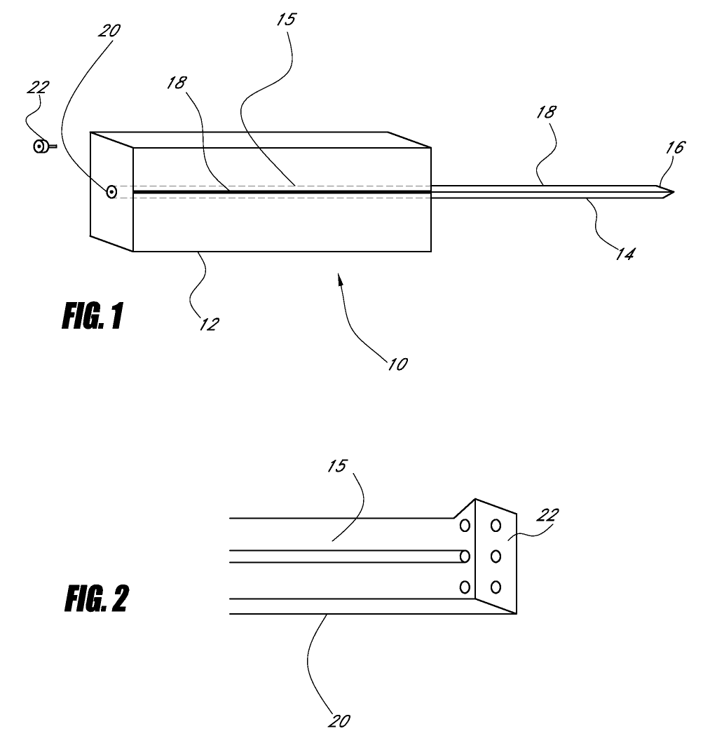

In some embodiments, the new device comprises a cannulated, fenestrated awl for use to augment micro-fracture surgery. FIG. 1 schematically illustrates an embodiment of the awl that comprises an awl body 10 having a handle 12 and a shaft 14. The awl body 10 extends along a generally longitudinal axis from a proximal end 13 of the handle 12 to an awl tip 16 at a distal end of the shaft 14. The handle 12 may be configured to be grasped by one or more hands of a user in order to maneuver the shaft 14 during a surgical procedure. The handle 12 shown in FIG. 1 is depicted as square-shaped but may be any convenient shape including but not limited to round, rectangular, or polygonal. In some embodiments, the handle 12 includes a textured and/or contoured surface to provide a suitable grip for the user. The handle 12 may have a length in a range from about 2-24 inches, more preferably 3-8 inches.

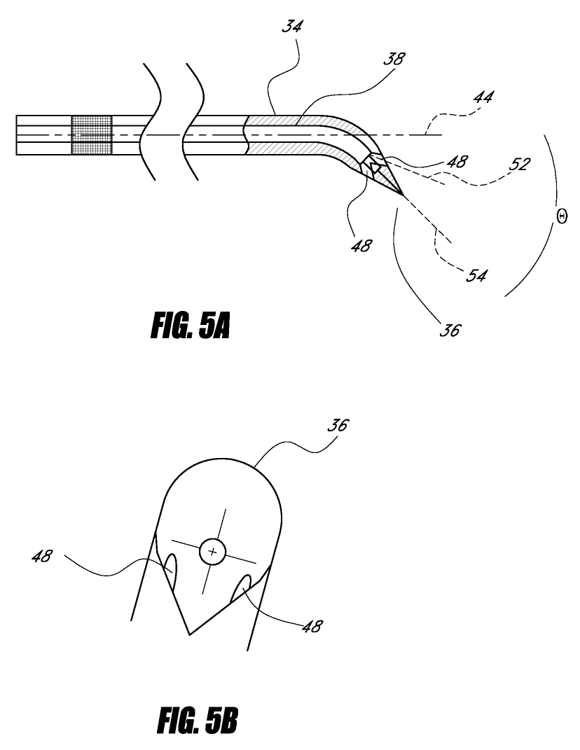

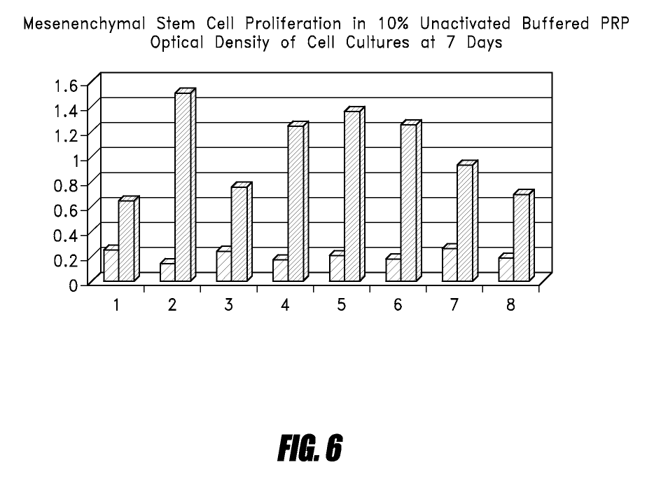

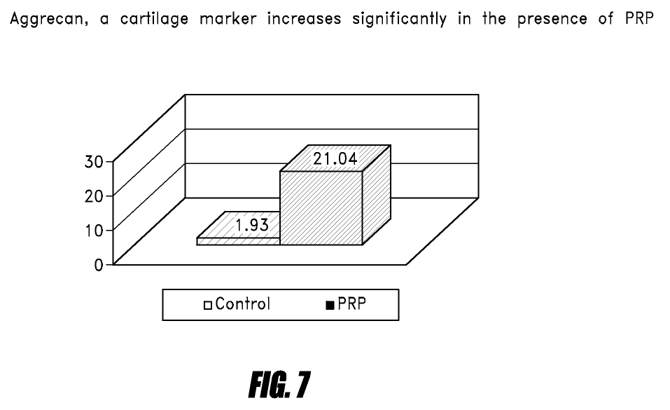

The shaft 14 extends from a distal end of the handle 12 to the awl tip 16. The shaft 14 may be any convenient length but preferably from 2-24 inches, and more preferably, about 5-12 inches. The shaft 14 may be substantially straight as shown, for example, in FIG. 1. In some embodiments, at least a portion of the shaft 14 is curved (see, e.g., the embodiments in FIGS. 4 and 5). The shaft 14 may have any suitable cross-sectional shape such as, for example, circular, oval, polygonal, etc. As schematically shown in FIG. 1, the shaft 14 may have a transverse width that tapers from a proximal end of the shaft 14 towards the awl tip 16. In other embodiments, the transverse width of the shaft 14 is substantially uniform away from the awl tip 16 (see, e.g., FIGS. 4 and 5).

In some embodiments, the awl body 10 is formed as a substantially integral unit. In other embodiments, the handle 12 and the shaft 14 are formed separately and then attached to each other. For example, the handle 12 and the shaft 14 may be attached by welds, adhesives, and/or mechanical coupling (e.g., mutually engaging threaded portions). The awl may be made out of any suitable material(s) including but not limited to metal, bone, ceramic, plastic or rubber or any number of potential materials based on the tissue that needs to be treated. In a preferred embodiment, the awl shaft 14 is constructed of a biocompatible material such as a metal, and the awl handle 12 is constructed of hard plastic. Preferably, the awl is sufficiently durable so that the awl can be manipulated to facilitate penetration of tissues such as cartilage and/or bone. For example, the awl may be formed from sufficiently hard and durable materials that the awl substantially resists deformation when placed under tensile and/or compressive forces (e.g., when struck with a mallet during a surgical procedure). In some preferred embodiments, the awl is disposable. Alternatively, selected parts of the awl may be disposable such as, for example, the entire shaft 14 including the tip 16 and/or the tip 16 alone. In embodiments where the shaft 14 (or portions thereof) are disposable, the shaft 14 advantageously may be configured to be readily attached and/or detached from the handle 12 such as by use of a threaded portion that can engage a complimentary threaded recess in the handle 12.

In certain preferred embodiments, the awl is cannulated. For example, at least a portion of the awl body 10 may include a lumen 15. In some embodiments, the lumen 15 extends from the proximal end 13 of the handle 12 to the awl tip 16. The lumen 15 may be disposed substantially along the longitudinal axis of the awl body 10. The lumen 15 has a cross-sectional area, which may be substantially uniform along the length of the lumen 15. In some embodiments, the cross-sectional area of the lumen 15 may vary along the length of the lumen 15, for example, by decreasing toward the awl tip 16. The shape of the cross-section of the lumen 15 may be substantially circular, oval, polygonal, or any other suitable shape. In a preferred embodiment, the lumen 15 is substantially circular in cross-section and has a diameter in a range from about 1 mm to 20 mm or more. In some embodiments, the awl includes two or more lumens.

The cannulated interior may optionally include a removable insert such as a plunger to push the material down the interior shaft of the device (not shown). In some embodiments, the awl includes an attachment point 20 for a syringe or catheter that may be used to transfer a fluid (such as a bioactive material) through the lumen 15. The attachment point 20 may be open or closed by use of, for example, a removable plug 22. As illustrated in FIG. 1, the attachment point 20 may be disposed on the proximal end 13 of the handle. In other embodiments, the attachment point 20 may be disposed elsewhere on the awl such as, for example, along a side of the handle 12 or along the shaft 14. In some embodiments, two or more attachment points may be used. In certain embodiments, the lumen 15 extends from the attachment point 20 to the awl tip 16.

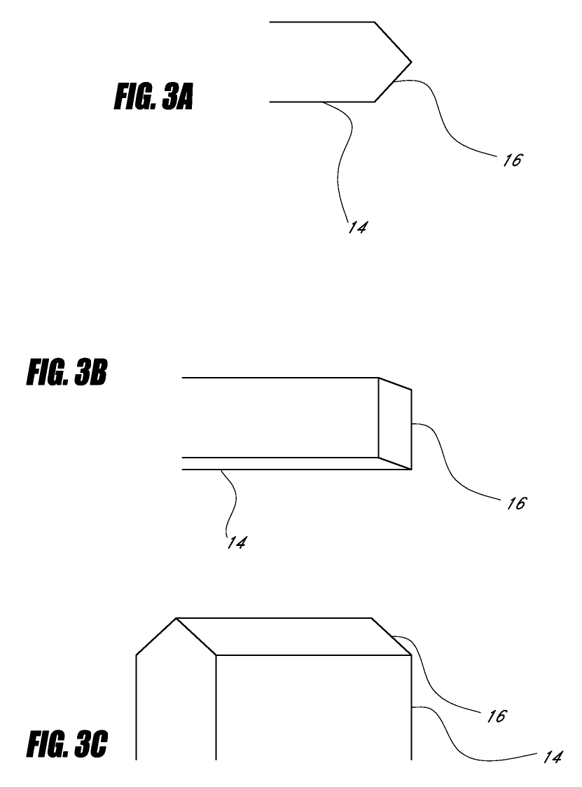

FIG. 2 schematically illustrates a view of part of the awl shaft 14 adjacent the awl tip 16. The distal end of the lumen 15 may include one or more openings at and/or near the awl tip 16 to permit fluid to flow from the tip 16. In preferred embodiments, the awl tip 16 may have multiple openings, preferably two, more preferably three, yet more preferably four, yet more preferably five, yet more preferably six. In some preferred embodiments, the awl tip 16 is perforated like a sieve (not shown). In some embodiments the awl tip 16 has a single opening. The openings may be formed at the distal end of the awl tip 16 and/or along the sides of the shaft 14 near the tip 16.

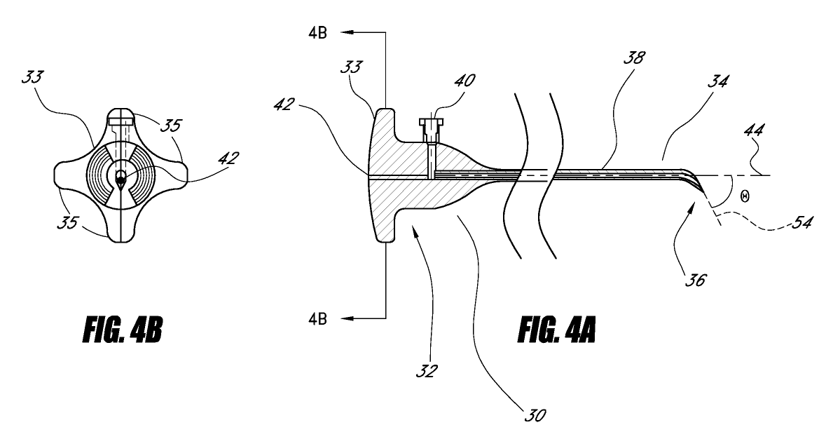

The awl tip 16 may be shaped to enable various functionalities. For example, the awl tip 16 may be pointed (see, e.g., FIG. 3a) or may be flat-bladed like a scraper (see, e.g., FIGS. 3b and 3c). The shape of the awl tip 16 advantageously may have any shape that is capable of creating a cavity in tissue suitable for introduction of a fluid material. The awl tip 16 may be configured to be removable from the distal end of the shaft 14. For example, the awl tip 16 may comprise a threaded portion configured to engage a complimentary threaded portion at the distal end of the shaft 14.

FIG. 4A schematically illustrates a side view of another embodiment of an awl 30. In this embodiment, the awl 30 includes a handle 32 and a shaft 34 terminating at an awl tip 36. In this embodiment, the proximal end of the handle 32 comprises a knob 33 that facilitates movement of the awl 30. The knob 33 may have a size and shape that permit the awl 30 to be easily grasped and manipulated so that the tip 36 may be inserted into tissue. For example, FIG. 4B is an end view of the awl 30 that shows an embodiment of the knob 33 having four transverse prongs 35. In other embodiments, two, three, five, six, or more prongs may be used. As discussed above with reference to FIG. 3a and FIG. 3b, the awl tip 36 may be shaped, e.g., pointed (FIG. 3a) or flat bladed like a scraper (FIG. 3b). In some embodiments, any tip capable of creating a cavity for introduction of a fluid material can be used.

The awl 30 may be cannulated. For example, the embodiment shown in FIG. 4A comprises a lumen 38 that extends from a side port 40 to the awl tip 16. The side port 40 may be configured to engage a syringe and/or a catheter for example via a luer lock. Fluid, such as a bioactive material, may be introduced through the side port 40 and may exit through one or more openings at or near the awl tip 36. In some embodiments, the lumen 38 additionally or alternatively extends through the handle 32 to an attachment point 42 at the proximal end of the handle 32, which may be configured to engage a syringe and/or a catheter (e.g., via a luer lock). In such embodiments, fluids may be introduced at the attachment point 42 and/or the side port 40. Fluids introduced at the attachment point 42 and the side port 40 may comprise the same or different material.

As shown in FIG. 4A, in some embodiments, the awl tip 36 may be angled at an angle θ with respect to a longitudinal axis 44 of the shaft 34. The angle θ may be fixed or variable in different embodiments. The angle θ may be any convenient angle, preferably between 0°-120°, yet more preferably, between 30°-60°. FIGS. 5A and 5B are closeup views schematically illustrating an embodiment of the awl tip 36 in more detail. In this embodiment, the angle θ is about 45 degrees. The lumen 38 is disposed substantially along the longitudinal axis of the shaft 34 and is angled to join the tip 36. The distal end of the lumen 38 terminates in two openings 48 that permit output of fluid at the surgical site. In other embodiments, a different number of openings 48 may be utilized such as, for example, one, three, four, five, six, or more. As described above, in some embodiments, the tip 36 may be perforated (like a sieve). The openings 48 may define an exit path 52 for the fluid that flows through the lumen 38. The fluid exit path 52 may be angled with respect to awl tip axis 54. The angle between the exit path 52 and the tip axis 54 may be in a range from about 0°-45° and is about 30° in one embodiment. Other angles between 0° and 180° may be used.

The tissue may be selected from one of the follow types but is not limited to these specific tissues: connective tissue, cardiac muscle or tissue, spinal tissue (including nerves, spinal cord, disc or vertebral bodies), internal organs (including pancreas, lungs, liver, intestines, bladder or other solid organ tissue), skin tissue, brain tissue, vascular tissue (including veins, arteries or lymphatic tissue), ocular, ear, nose or throat tissue, or other hematologic, endocrine or integumentary tissue. In a preferred embodiment, the tissue is connective tissue. Connective tissue includes but is not limited to cartilage, ligament, tendons, bone and spinal tissue.

The tissue may be human or other non-human animal tissue. In preferred embodiments, the device is used in human and animal patients, particularly veterinary animals such as dogs, cats, horses, pigs, sheep, and cows. More preferably, the device is used on a human subject.

In a preferred embodiment, the device may be used to treat a full or partial thickness cartilage injury inside a joint in a human or animal. The device is specifically and uniquely designed to initially penetrate tissue and then be able to deliver a bioactive substance into that area of penetration.

Any bioactive substance can be delivered via the device. These may include but are not limited to platelet rich plasma (PRP), stem cells, bone marrow cells, bone marrow aspirate, drugs, individual growth factors or synthetic materials.

In a preferred embodiment, the bioactive substance may include a biocompatible composition that comprises unactivated platelets, activated platelets, platelet releasate(s), and/or the like. In a preferred embodiment, the bioactive substance comprises platelet-rich plasma (PRP).

PRP is an enriched platelet-containing mixture, isolated from whole blood, which is resuspended in a small volume of plasma. While whole blood may contain about 95% red blood cells, about 5% platelets and less than 1% white blood cells, PRP may contain 95% platelets with 4% red blood cells and 1% white blood cells. PRP can be combined with activating agents such as thrombin or calcium which activate the platelets to release their contents such as cytokinins and other growth factors. In some preferred embodiments, PRP is used without activation.

The term “PRP” as used herein is a broad term which is used in its ordinary sense and is a concentration of platelets greater than the peripheral blood concentration suspended in a solution of plasma, with typical platelet counts ranging from 500,000 to 1,200,000 per cubic millimeter, or even more. PRP is formed from the concentration of platelets from whole blood, and may be obtained using autologous, allogenic, or pooled sources of platelets and/or plasma. PRP may be formed from a variety of animal sources, including human sources.

Platelets are cytoplasmic portions of marrow megakaryocytes. They have no nucleus for replication; the expected lifetime of a platelet is some five to nine days. Platelets are involved in the hemostatic process and release several initiators of the coagulation cascade. Platelets also release cytokines involved with initiating wound healing. The cytokines are stored in alpha granules in platelets. In response to platelet to platelet aggregation or platelet to connective tissue contact, as would be expected in injury or surgery, the cell membrane of the platelet is “activated” to secrete the contents of the alpha granules. The alpha granules release cytokines via active secretion through the platelet cell membrane as histones and carbohydrate side chains are added to the protein backbone to form the complete cytokine. Platelet disruption or fragmentation, therefore, does not result in release of the complete cytokine.

A wide variety of cytokines are released by activated platelets. Platelet derived growth factor (PDGF), transforming growth factor-beta (TGF-b), platelet-derived angiogenesis factor (PDAF) and platelet derived endothelial cell growth factor (PD-ECGF) and insulin-like growth factor (IGF) are among the cytokines released by degranulating platelets. These cytokines serve a number of different functions in the healing process, including helping to stimulate cell division at an injury site. They also work as powerful chemotactic factors for mesenchymal cells, monocytes and fibroblasts, among others. For the purposes of this patent, the term “releasate” refers to the internal contents of the platelet, including cytokines, which have the potential to affect another cells' function.

In another embodiment, the inventive platelet composition may comprise releasate from platelets, in addition to platelets themselves. The releasate comprises the various cytokines released by degranulating platelets upon activation. Many activators of platelets exist; these include calcium ions, thrombin, collagen, epinephrine, and adenosine diphosphate. Releasates according to the invention may be prepared according to conventional methods, including those methods described in U.S. Pat. No. 5,165,938 to Knighton, and U.S. Pat. No. 5,599,558 to Gordinier et al.

One disadvantage of conventional releasate strategies associated with the use of PRP as platelet gel (PG) is the use of thrombin as a preferred activator. In particular, much thrombin used in PG is bovine thrombin, which can create problems due to contamination issues regarding Creutzfeldt-Jakob disease. Many bovine materials are suspect due to possible prion contamination, and so use of bovine thrombin is disfavored in surgery. Human pooled thrombin is likewise disfavored due to the potential of contamination with various materials such as viruses, prions, bacteria and the like. Recombinant human thrombin might also be used, but is quite expensive.

In preferred embodiments, an activator is not used with PRP as the bioactive substance. Collagen, a major component of connective tissues, is a strong activator of platelets. Thus, when the inventive platelet composition is introduced into and/or around connective tissue, platelets in the platelet composition may bind to the collagen and then be activated. This reduces or eliminates the need for administering an exogenous activator such as thrombin. The disadvantages of thrombin use have been noted above. Other strong activators, such as calcium ions, can cause severe pain, unintentional clotting, and other undesirable side effects. Thus, in a preferred embodiment of the invention, PRP is used as the bioactive substance with no or substantially no exogenous activator added, or in the preparation of the bioactive PRP composition. Of course, exogenous activators may still be employed if a physician determines that they are medically necessary or desirable.

The platelet composition may be prepared using any conventional method of isolating platelets from whole blood or platelet-containing blood fractions. These include centrifugal methods, filtration, affinity columns, and the like. If the platelet composition comprises PRP, then conventional methods of obtaining PRP, such as those disclosed in U.S. Pat. Nos. 5,585,007 and 5,788,662 both to Antanavich et al., incorporated herein by reference in their entirety, may be utilized.

In some embodiments, PRP is used as the bioactive substance with co-administration of one or more of the ingredients selected from thrombin, epinephrine, collagen, calcium salts, pH adjusting agents.

Adjusting the pH of platelet compositions has been used to prolong the storage time of unactivated platelets, as disclosed in U.S. Pat. No. 5,147,776 to Koerner, Jr. and U.S. Pat. No. 5,474,891 to Murphy, incorporated by reference herein. pH may be adjusted using a variety of pH adjusting agents, which are preferably physiologically tolerated buffers, but may also include other agents that modify pH including agents that modify lactic acid production by stored platelets. Especially useful are those pH adjusting agents that result in the pH of the platelet composition becoming greater than or equal to physiological pH. In an embodiment, the pH adjustment agent comprises sodium bicarbonate. Physiological pH, for the purposes of this invention, may be defined as being a pH ranging from about 7.35 to about 7.45. pH adjusting agents useful in the practice of this invention include bicarbonate buffers (such as sodium bicarbonate), calcium gluconate, choline chloride, dextrose (d-glucose), ethylenebis(oxyethylenenitrilo)tetraacetic acid (EGTA), 4-(2-hydroxyethyl)-1-piperazineethanesulfonic acid (HEPES), maleic acid, 4-morpholinepropanesulfonic acid (MOPS), 1,4-piperazinebis(ethanesulfonic acid) (PIPES), sucrose, N-tris(hydroxymethyl)methyl-2-aminoethanesulfonic acid (TES), tris(hydroxymethyl)aminomethane (TRIS BASE), tris(hydroxymethyl)aminomethane hydrochloride (TRIS.HCl), and urea. In a preferable embodiment, the pH adjusting agent is a bicarbonate buffer, more preferably, sodium bicarbonate.

In some embodiments, growth factors or growth factor inhibitors, small molecule pharmaceuticals such as NSAIDS, steroids, and anti-infective agents may be introduced using the microfracture awl device.

The following description may be applied to any tissue. An area of a cartilage defect would initially be identified by x-ray, MRI, ultrasound or other imaging modality. The preferred imaging study used is determined by the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging and ultrasound techniques. The patient may also assist in locating the site of tissue injury or damage by pointing out areas of particular pain and/or discomfort. Other ways of finding the area of the defect include but are not limited to direct visual inspection and palpation.

Once the area of the cartilage defect has been identified, the device would be inserted into that area either under direct vision or via robotic or imaging guidance. Once inside the lesion area, it would be used to create a cavity. In the case of the knee, this would be a cavity in the subchondral bone leading directly to the bone marrow. A syringe or other delivery system would then be attached to the cannulated device to directly deliver bioactive substances into the cavity to affect a change in the tissue.

In the case of the cartilage defect, one possible bioactive substance is PRP. In some embodiment, other substances such as drugs, small molecules, viruses for gene therapy or other substances are applied to the cavity by the inventive device. In some embodiments, the device is used multiple times in a specific defect to create multiple cavities and repeatedly deliver these bioactive substances.

The device may optionally include a plunger to put through the cannula to push the bioactive material into the cavity created by the awl.

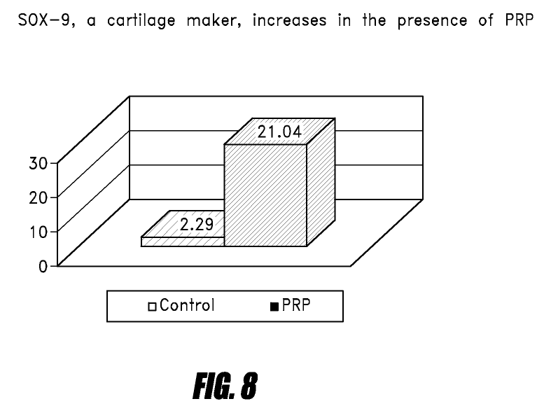

Importantly, it has been found by the inventor that platelet rich plasma (PRP) delivered to mesenchymal stem cells increases their proliferation and leads them toward chondrogenic differentiation (FIG. 6). Using this inventive device, PRP could be delivered to the defect and lead to improved fill and therefore better clinical outcomes. Specifically, PRP has been shown to increase mesenchymal stem cell proliferation five fold in seven days. Cartilage markers such as aggrecan (FIG. 7) and SOX-9 (FIG. 8) are significantly increased in the presence of PRP.

### EXAMPLES

### Example 1

PRP was prepared using a centrifuge unit made by Harvest (Plymouth, Mass.). (Similar units are available as The Biomet GPS system, the Depuy Symphony machine and the Medtronic Magellan machine.) Approximately 55 cc of blood was drawn from the patient using a standard sterile syringe, combined with 5 cc of a citrate dextrose solution for anticoagulation, and then spun down to isolate the platelets according to the manufacturer's protocol. These platelets were then resuspended in approximately 3 cc of plasma. The resulting platelet rich plasma solution (PRP) was quite acidic and was neutralized with using approximately 0.05 cc of an 8.4% sodium bicarbonate buffer per cc of PRP under sterile conditions to approximately physiologic pH of 7.4. The PRP was not activated through addition of exogenous activators. This PRP composition is referred to herein as autologous platelet extract (APEX).

### Example 2

Fifty cc of whole blood is drawn from a patient, and then prepared according to the method of Knighton, U.S. Pat. No. 5,165,938, column 3. The PRP is activated according to Knighton using recombinant human thrombin. The degranulated platelets are spun down and the releasate containing supernatant is recovered. The releasate may be optionally pH adjusted to a pH of 7.4 using sodium bicarbonate buffer.

### Example 3

Thirty ml of whole blood were drawn from a patient. A platelet composition was prepared according to Example 1 of U.S. Pat. No. 5,510,102 to Cochrum, incorporated herein by reference in its entirety, except that no alginate is added to the platelet composition.

### Example 4

### Meniscus Tissue

An area of defective meniscus tissue is initially identified by x-ray, MRI, ultrasound or other imaging modality. The defect may be a tear or a tissue degeneration. An area of pain or swelling may also be used to determine the site of a cartilage lesion. Other ways of finding the area of the defect include but are not limited to direct visual inspection and palpation.

Once the area of defective meniscus tissue is identified, a local, spinal or general anesthetic is administered. The microfracture awl device is inserted into the identified area either under direct vision or via robotic or imaging guidance. Once inside the lesion area, the microfracture awl device is used to create a cavity. The inner cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 5

### Spinal Disc Tissue

An area of defective spinal tissue is initially identified by x-ray, MRI, ultrasound or other imaging modality. The defective disc tissue may be a herniated disc or degenerative disc disease. An area of pain or swelling may also be used to determine the site of a the lesion. Other ways of finding the area of the defect include but are not limited to direct visual inspection and palpation.

Once the area of defective spinal disc tissue is identified, a local, spinal or general anesthetic is administered. The microfracture awl device is inserted into the identified area either under direct vision or via robotic or imaging guidance. Once inside the lesion area, the microfracture awl device is used to create a cavity. The inner cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 6

### Spinal Vertebral Bone

An area of defective spinal vertebral bone is initially identified by x-ray, MRI, ultrasound or other imaging modality. The defective spinal vertebral bone may be a compression fracture or other disorder. An area of pain or swelling may also be used to determine the site of a the lesion. Other ways of finding the area of the defect include but are not limited to direct visual inspection and palpation.

Once the area of defective spinal vertebral bone is identified, a local, spinal or general anesthetic is administered. The microfracture awl device is inserted into the identified area either under direct vision or via robotic or imaging guidance. Once inside the lesion area, the microfracture awl device is used to create a cavity. The inner cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 7

### Fascia

An area of defective fascial tissue is initially identified by x-ray, MRI, ultrasound or other imaging modality. The defective fascial tissue may be a tight fascia, for example. An area of pain or swelling may also be used to determine the site of a the lesion. Other ways of finding the area of the defect include but are not limited to direct visual inspection and palpation.

Once the area of defective fascia is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 8

### Joint

A defective joint such as a knee, shoulder, elbow or ankle is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of defective joint tissue is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 9

### Tendon

A patient presents with tendinitis, tendinosis, or a tendon tear. The defective tendon is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of defective tendon is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

### Example 10

### Bone

A patient presents with osteoporosis or a bone fracture. The defective bone is initially identified by x-ray, MRI, ultrasound or other imaging modality. An area of pain or swelling may also be used to determine the site of a the lesion. Other ways of finding the area of the defect include but are not limited to direct visual inspection and palpation.

Once the area of fractured bone or osteoporosis is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, bone marrow aspirate and/or bone marrow cells. In preferred embodiments, the bioactive substance includes PRP. The inner cannula is closed and the awl is used to compress material into the treated area. The awl is removed and the procedure may be repeated multiple times to treat the entire affected area. This procedure may be combined with other procedures to repair the fractured bone. Following the procedure, appropriate clinical and/or imaging modalities are implemented to follow the result of the procedure.

### Example 11

### Neoplastic Tissue

A patient presents with a benign or malignant tumor or a bone cyst. The defective bone is initially identified by x-ray, MRI, ultrasound or other imaging modality in combination with physical examination, optionally in combination with blood or tissue work such as a biopsy to characterize the nature of the disease. An area of pain or swelling may also be used to determine the site of a the lesion. Other ways of finding the area of the defect include but are not limited to direct visual inspection and palpation.

Once the diseased area is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. This procedure may be combined with other procedures to treat the tumor or cyst. Following the procedure, appropriate clinical and/or imaging modalities are implemented to follow the result of the procedure.

### Example 12

### Infected Tissue

A patient presents with an infection such as a superficial wound or a deep abscess. The infected tissue is initially identified by x-ray, MRI, ultrasound or other imaging modality in combination with physical examination, and/or clinical blood or tissue work to characterize the nature of the disease. An area of pain or swelling may also be used to determine the site of the infection. Other ways of finding the area of the defect include but are not limited to direct visual inspection and palpation.

Once the infected area is identified, a local, spinal or general anesthetic is administered. The microfracture awl device is inserted into the identified area either under direct vision or via robotic or imaging guidance. Once inside the lesion area, the microfracture awl device is used to create a cavity. The cannula of the awl is opened and a syringe or other delivery system is attached to the cannulated microfracture awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. This procedure may be combined with other procedures to treat the infection. Following the procedure, appropriate clinical and/or imaging modalities are implemented to follow the result of the procedure.

### Example 13

### Cardiac Muscle Tissue

A patient presents with an myocardial infarction, congestive heart failure or valve disease. An area of pathologic cardiac muscle or defective valve is identified using imaging techniques such as x-ray, MRI, blood work, and/or physical examination. Once the area is identified, a general anesthetic is administered. The awl device is inserted into the identified area of cardiac tissue or defective valve either under direct vision or via robotic or imaging guidance. Either endovascular, minimally invasive or open surgical techniques may be used. Once inside the lesion area, the awl device is used to create a cavity. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. This procedure may be combined with other procedures. The procedure is combined with appropriate drugs or chemotherapy agents. Following the procedure, appropriate clinical and/or imaging modalities are implemented to follow the result of the procedure.

### Example 14

### Skin Tissue

A patient presents with a melanoma, other skin cancer or skin lesion. The area of the skin lesion is identified visually by physical examination. Clinical evaluation of tissue samples may also be conducted to characterize the disease.

Once the area is identified, an anesthetic is administered, generally a local anesthetic is sufficient. The awl device is inserted into the identified area of skin to penetrate the skin, create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

### Example 15

### Pancreas

A patient presents with symptoms of diabetes. A pathological area of the pancreas is identified using physical examination, clinical blood or tissue work, or imaging techniques such as x-ray or MRI. Clinical evaluation of tissue samples may also be conducted to characterize the disease.

Once the area is identified, a local, spinal or general anesthetic is administered. The awl device is inserted into the identified area of skin to penetrate the organ, create a cavity and deliver bioactive substances. Either open techniques or endovascular, minimally invasive surgical techniques may be used. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. The procedure is combined with appropriate drugs or chemotherapy agents. Following the procedure, appropriate clinical and/or imaging modalities are implemented to follow the result of the procedure.

### Example 16

### Brain/Nervous Tissue

A patient presents with a brain tumor or spinal cord injury. The area of pathological tissue is identified using physical examination or imaging techniques such as x-ray or MRI. Clinical evaluation of tissue or blood samples may also be conducted to characterize the disease.

Once the area is identified, a local, spinal or general anesthetic is administered. The awl device is inserted into the identified bone or nerve sheath to create a cavity and deliver bioactive substances. Either open techniques or endovascular, minimally invasive surgical techniques may be used. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

### Example 17

### Ocular, Ear, Nose and Throat Tissue

A patient presents with a disease of the eye, ear, nose or throat such as sinus lesions, glaucoma or ear lesions. The area of pathological tissue is identified using physical examination or imaging techniques such as x-ray or MRI. Clinical evaluation of tissue or blood samples may also be conducted to characterize the disease.

Once the area is identified, a local, spinal or general anesthetic is administered. The awl device is inserted into the identified eye, ear, nose or throat tissue to create a cavity and deliver bioactive substances. Either open techniques or endovascular, minimally invasive techniques may be used. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics or chemotherapy agents. Following the procedure, appropriate clinical and/or imaging modalities are implemented to follow the result of the procedure.

### Example 18

### Dental Applications

The device may be used for a variety of dental procedures including but not limited to bone grafting, periodontal work or cosmetic work. The area of abnormality is identified using physical examination or imaging techniques such as x-ray or MRI. Once the area is identified, a local, spinal or general anesthetic is administered. The awl device is inserted into the identified tissue to create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. The inner cannula is closed and the awl is used to compress material into the treated area. The awl is removed and the procedure may be repeated multiple times to treat the entire affected area. The procedure is combined with appropriate drugs, such as antibiotics. Following the procedure, appropriate clinical and/or imaging modalities are implemented to follow the result of the procedure.

### Example 19

### Hair Transplant

The device is used to deliver bioactive material to enhance hair growth. An area of thinning hair growth or baldness is identified using physical examination or imaging techniques such as x-ray or MRI. Once the area is identified, a local anesthetic is administered to numb the area. The awl device is inserted into the identified area of scalp or skin to create a cavity and deliver bioactive substances. The cannula of the awl is opened and a syringe or other delivery system is attached to the cannulated awl device to directly deliver bioactive substances into the cavity to affect a change in the tissue. The bioactive substance may be PRP, one or more pharmaceutical drugs, growth factors, stem cells, and/or bone marrow cells. In preferred embodiments, the bioactive substance includes PRP. A hair follicle is implanted into the cavity created by the awl. The surgical procedure is completed according to standard protocol. The inner cannula is closed and the awl is used to compress material into the treated area. The awl is removed and the procedure may be repeated multiple times to treat the entire affected area. Following the procedure, appropriate clinical and/or imaging modalities are implemented to follow the result of the procedure.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A device comprising a tapered elongated member having a proximal end, a central longitudinal axis, and an awl shaped distal end, wherein the proximal end comprises a handle for manipulation of the device and an access port comprising a luer lock on a side of the handle for the introduction of a fluid, and wherein the awl shaped distal end comprises an outlet port for discharge of the fluid, and wherein the access port is adapted to be in fluid communication with the outlet port through an internal channel disposed within the elongated member, the internal channel forming a hollow length extending throughout at least a part of the tapered elongated member, and wherein the tip of the awl shaped distal end is angled with respect to the central longitudinal axis.

2. A device comprising a tapered elongated member having a handle at a proximal end and a shaft comprising an awl shaped distal end, wherein the elongated member comprises an internal channel disposed along a longitudinal axis within the elongated member, the internal channel forming a hollow length extending along at least a portion of the longitudinal axis, the internal channel having at least one input and an output at the awl shaped distal end.

3. The device of claim 2, wherein the output comprises multiple openings.

4. The device of claim 2 wherein the hollow length extends along substantially the entire length of the longitudinal axis.

5. The device of claim 2, wherein the internal channel is adapted for delivery of a liquid.

6. The device of claim 2, comprising a single input at the proximal end.

7. The device of claim 2, comprising a single input at a side of the proximal end.

8. The device of claim 2, wherein at least a portion of the shaft is curved.

9. The device of claim 2, wherein the awl tip comprises a point.

10. The device of claim 2, wherein the awl tip comprises a scraper.

11. The device of claim 2, further comprising a plunger adapted to engage the internal channel.

12. The device of claim 2, further comprising an attachment point at the input, the attachment point configured to receive a syringe.

13. The device of claim 2, wherein the awl shaped distal end and the longitudinal axis form an angle therebetween.

14. The device of claim 13, wherein the angle is in a range from about 0° to about 30°.

15. The device of claim 13, wherein the angle is in a range from about 30° to about 60°.

16. A method of treating an injury, wear or defect in an individual comprising:
(a) identifying an area of injury, wear or defect;
(b) inserting the device of claim 1 into the identified area;
(c) creating a cavity in the area using the awl shaped distal end of the device;
(d) attaching a delivery system to the access port for delivery of a bioactive substance;
(e) delivering the bioactive substance through the internal channel and into the identified area;
(f) closing the access port on the device;
(g) compressing the bioactive substance into the identified area; and
(h) removing the device.

17. The method of claim 16, further comprising repeating steps (b) through (h).

18. The method of claim 16, wherein the area of injury, wear or defect comprises a tissue selected from the group consisting of connective tissue, cardiac muscle or tissue, spinal tissue, internal organs, skin tissue, brain tissue, vascular tissue, ocular, ear, nose, and throat tissue.

19. The method of claim 18, wherein the spinal tissue is selected from the group consisting of nerves, spinal cord, disc material and vertebral bodies.

20. The method of claim 18, wherein the internal organ is selected from the group consisting of pancreas, lungs, liver, intestines, and bladder.

21. The method of claim 18, wherein the vascular tissue is selected from the group consisting of veins, arteries and lymphatic tissue.

22. The method of claim 18, wherein the connective tissue is selected from the group consisting of articular cartilage, meniscus cartilage, ligament, tendons, fascia, bone and spinal tissue.

23. The method of claim 16, wherein the area of injury, wear or defect is identified by X-ray or MRI.

24. The method of claim 16, wherein the bioactive substance is selected from the group consisting of platelet rich plasma (PRP), stem cells, bone marrow cells, bone marrow aspirate, drugs, individual growth factors and synthetic materials.

25. The method of claim 24, wherein the bioactive substance is PRP.

26. The method of claim 25, wherein no exogenous activator is added to the PRP prior to delivery into the identified area.

27. The method of claim 25, wherein the PRP comprises platelets obtained from the individual.

28. The method of claim 24, further comprising the step of titrating the PRP to obtain a pH of about 7.3 to 7.5.

29. The method of claim 28, wherein the titration is performed using a bicarbonate buffer.

30. The method of claim 25, further comprising the step of mixing the PRP substantially simultaneously prior to delivery into the identified area, with one or more ingredients selected from the group consisting of thrombin, epinephrine, collagen, calcium salts, and pH adjusting agents.
